(19) United States
(12) Patent Application Publication
(10) Pub. No.: US 2010/0256343 A1
(43) Pub. Date: Oct. 7, 2010

(54) Immobilization particles for targeted removal of microorganisms and substances

(76) Inventor: Brian Christopher Lue, Mountain View, CA (US)

Correspondence Address: Brian Lue, 284 Mountain View Ave, Mountain View, CA, 94041, US

(21) Appl. No.: 12/660,459

(22) Filed: Feb. 26, 2010

Related U.S. Application Data
(60) Provisional application No. 61/208,629, filed on Feb. 26, 2009.
(60) Provisional application No. 61/212,375, filed on Apr. 11, 2009.

(51) Int. Cl. C07K 16/00

(52) U.S. Cl. 530/391.1

(57) ABSTRACT

Embodiments generally provide an apparatus and method for adhering microorganisms, chemical, and biopolymers to a substrate for selective immobilization and later removal from the host organism such as a human or animal with minimal absorption and toxicity to the host. Embodiments provide for a method and apparatus control the microbial, chemical, and biopolymer compositions on the skin, upper and lower respiratory tracts, as well as the gastrointestinal system. Embodiments provide a method and apparatus to modulate the state of health and disease states of the host.

This patent application relates to a U.S. Provisional Application having Appl. No. 61/208,629 which was filed Feb. 26, 2009, from which priority is claimed under 35 USC §119(e), and which provisional application is incorporated herein in its entirety.

This patent application relates to a U.S. Provisional Application having Appl. No. 61/212,375 which was filed Apr. 11, 2009, from which priority is claimed under 35 USC §119(e), and which provisional application is incorporated herein in its entirety.

### TECHNICAL FIELDS

The field is pharmaceuticals and biological separation processes.

### BACKGROUND

The mixture of microorganisms in a person's gastrointestinal tract greatly affects a person's health. Some of the beneficial effects of those microorganisms aid in the digestion of food, create vitamins, sequester and neutralize toxic metals, create anti-cancer compounds, secrete beneficial enzymes, and prevent many pathogenic microorganisms from colonizing the gastrointestinal tract. From when a person is approximately one year old til when they are in their 50's to 60's, the composition bacteria population is mostly stable. A combination of genetics, bacterial exposure from the environment, and their diet help determine the strains and quantities of the bacteria that colonize their tract. For most of normal, healthy individuals, their microbial population, or microbiome, does not cause any problems. Unfortunately for others, their microbiome becomes dysfunctional and creates various chronic health problems. There are many triggers to make a dysfunctional microbiome. One common cause is the use of antibiotics and antifungals. Antibiotics and antifungals kill many kinds of bacteria and fungi, both helpful and hurtful. When antibiotics and/or antifungals are used, beneficial bystander bacteria and/or fungi get killed, the natural balance gets perturbed and the remaining beneficial bacteria and/or fungi can lose their ability to inhibit harmful ones. Also, certain antibiotics can change the behavior of normally present bacteria and make them harmful or more difficult for the immune system or antibiotics to target. For example, Penicillin G makes Proteus bacterium become cell wall deficient. Once in this form, many antibiotics cannot kill them.

Once the bacterial and/or fungal population gets perturbed by antibiotics and/or antifungals, the enzymes present in the gastrointestinal tract can change and the normal distribution of peptides seen by the immune system can change. If the wrong kinds of peptide sequences survive in sufficient concentrations, sequences that sufficiently resemble various molecules of the host's organ or other tissues, then autoimmune disease can initiate.

### SUMMARY

One or more embodiments of the present invention solve one or more of the above-identified problems by providing the means to immobilize a microbe or a chemical on the surface of a structure such that the fluid containing the microbe or chemical that is adjacent to the body's tissues, such as inside the gastrointestinal tract, can reach the immobilizing surfaces of structure to be disabled while the active immobilized microbes or chemicals remain out of contact with the body's tissues—to prevent a harmful immune or antibody reaction.

### BRIEF DESCRIPTION OF THE DRAWINGS

FIG. 1 shows a close-up of the surface of an immobilization particle with a covalently bound antibody. The spacers or larger substrate shape is not shown.

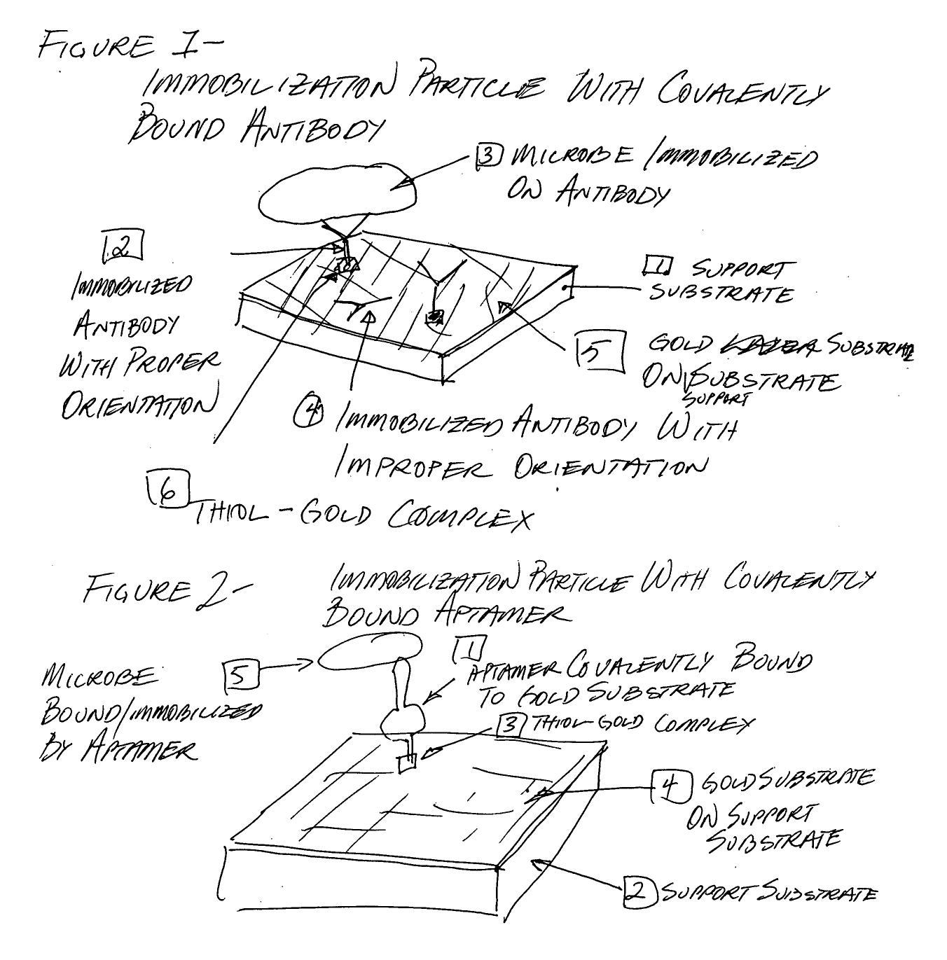

FIG. 2 shows a close-up of an immobilized particle with covalently bound aptamer

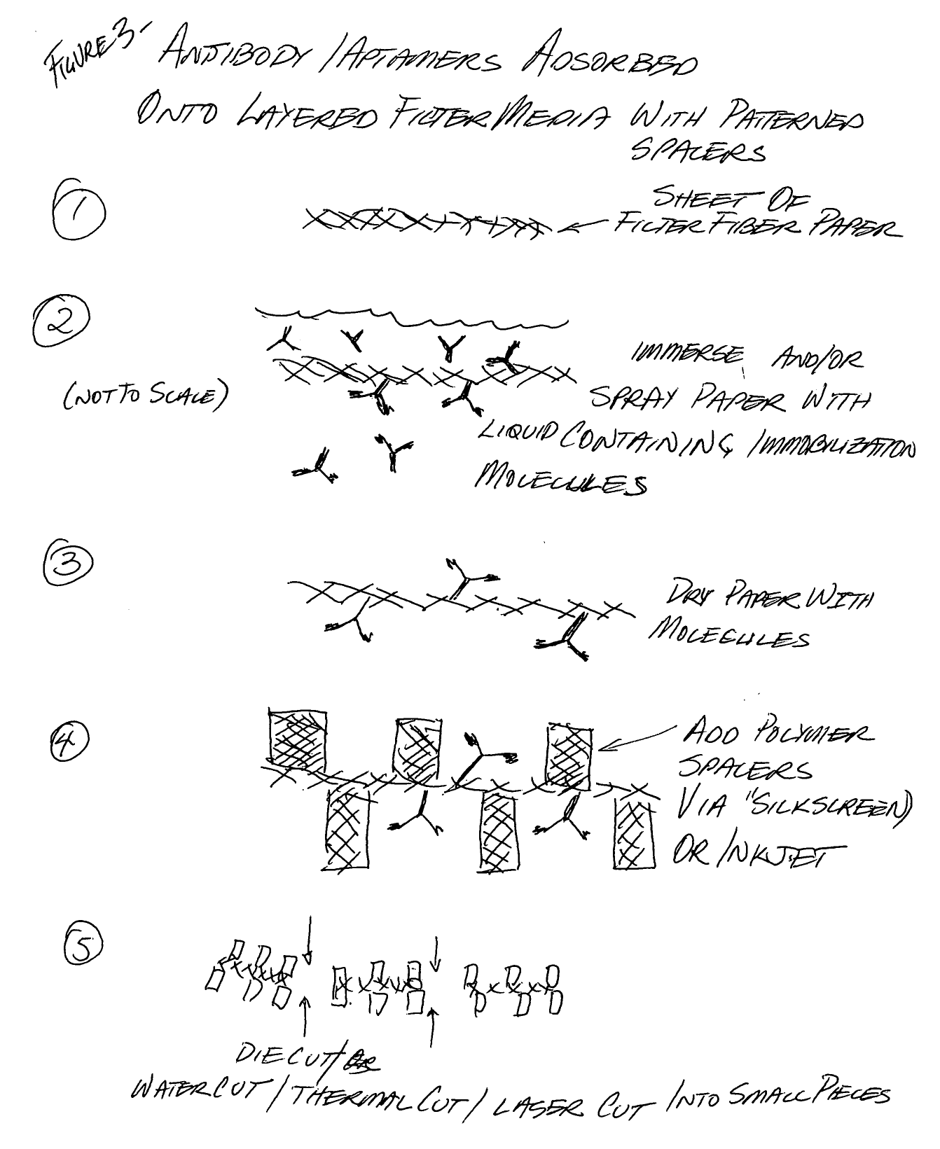

FIG. 3 shows antibodies and aptamers adsorbed onto layered filter media with patterned spacers

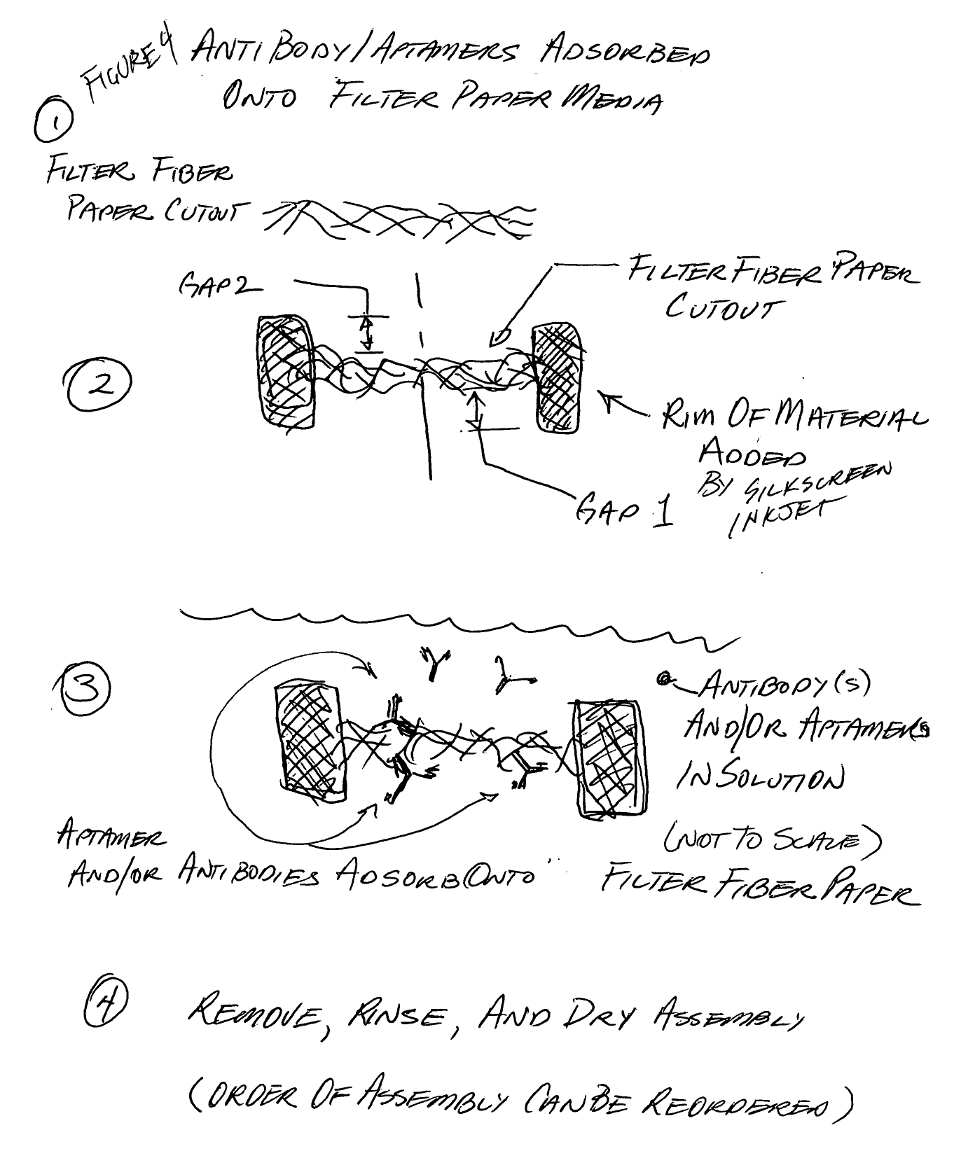

FIG. 4 shows antibodies and aptamers adsorbed onto filter paper media with edge spacers

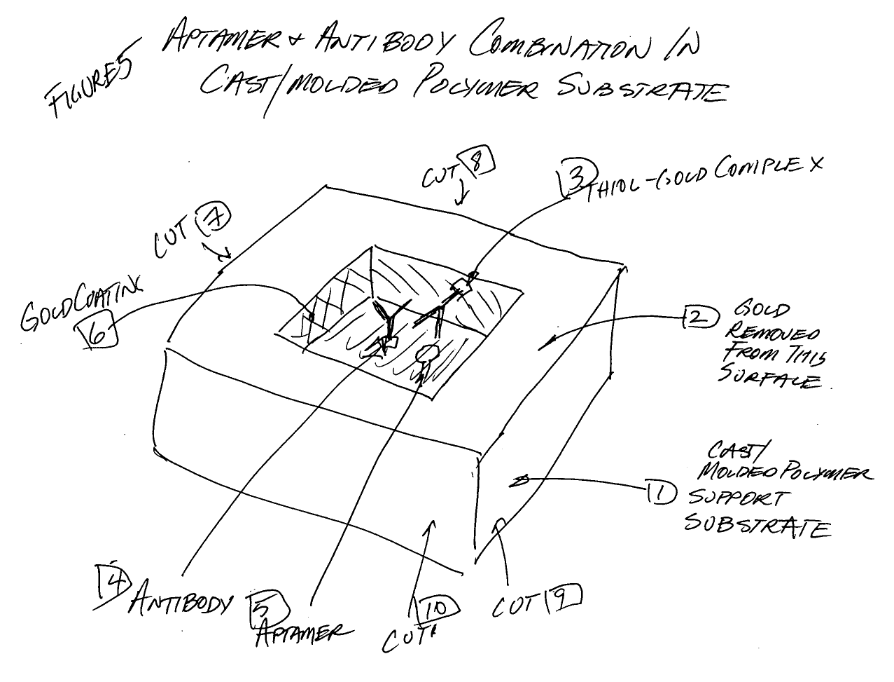

FIG. 5 shows an aptamer and antibody combination in cast/molded polymer substrate. Note one or more embodiments of this invention permit a mixture of aptamers and antibodies on immobilizing particles.

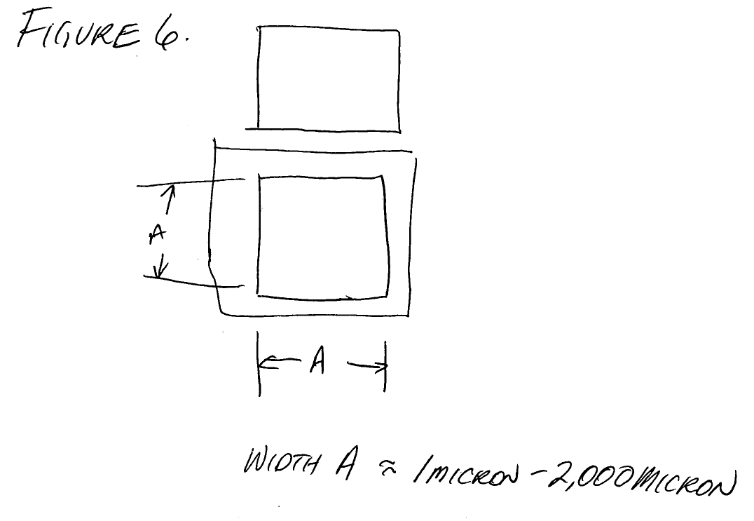

### DETAILED DESCRIPTION

One or more embodiments of this invention is a selectively immobilizes specific, targeted microbes or chemicals out of a person's body. The primary location where one or more embodiments of this invention would be most relevant is in the gastrointestinal tract. Unlike an antibiotic, in the case of a microbe, it can do this without killing the microbe so that the microbe doesn't break up into pieces which prevents an undesirable interaction with the immune system. One or more embodiments of this invention can remove triggers of autoimmune disease and its symptoms, peptide sequences that resemble portions of human tissue called mimics. It can also remove chemicals before they cause unwanted reactions, such as improper concentrations enzymes that can cause abnormal concentrations of peptide sequences that can cause and autoimmune reaction.

One or more embodiments of this invention involves at least two major components. Firstly, there is a substrate that is shaped to prevent an immobilized microbe or chemical from interacting with the host immune system.

Secondly, there is at least one type of immobilizing molecule that is attached to the substrate. There are two major types of immobilizing molecules, antibodies and aptamers. Antibodies are fairly large proteins (approx 150 kDa) that are created by living organisms that are capable of binding very specifically to proteins or other chemicals. The antibodies used for one or more embodiments of this invention are from standard monoclonal or polyclonal antibody production techniques. Because living organisms are needed to create antibodies, they are subject to batch-to-batch variations which results in an complicated government approval process. On the other hand, aptamers are chemically synthesized bits of single-stranded RNA or DNA molecules or peptides that are optimized by a complex sorting process. Examples of the sorting processes are, but not limited to are, Systematic Evolution of Ligands by Exponential Enrichment (SELEX) (Tuerk, et al. Systematic evolution of ligands by exponential enrichment: RNA ligands to bacteriophage T4 DNA polymerase. Science. 3 Aug. 1990 249: 505-510). The aptamers are typically 100 nucleotides long. The inventor has discovered that aptamers have several advantages over antibodies in the creation of one or more embodiments of the present invention. Since they are synthesized and do not require animals or organisms to manufacture, they have minimal batch-to-batch variation, they are non-toxic, relatively non-immunogenic, they bind to whole cell or molecular targets with similar affinity and specificity as antibodies, they are readily mass produced which makes them less expensive than antibodies, they are small so they less susceptible to steric interference and can even penetrate into the cell's interior. Also, DNA aptamers are more thermally stable than antibodies. From this point hereafter the combination of at least the substrate and immobilizing molecule will be called an immobilization particle.

### Substrate Design

In one or more embodiments of this invention, immobilization particles have a single or multiple concave cavities with the immobilizing molecule attached to and/or coating the concave surfaces. The concave surfaces are the preferred immobilization surfaces because when the particles immobilize the single celled organisms, parts of the organisms would avoid contact with the surfaces of the gastro-intestinal wall, or would be harder to reach by the tentacles of the dendritic cells. This lack of physical contact would prevent the immune system from being aware of the presence of that organism or its parts and an undesirable immune response if the patient has the potential for generating cross-reacting antibodies to the immobilized organisms that could attack other body tissues as in the case of autoimmunity. Coating the interior of concave surfaces also prevents cross-agglomeration between particles due to inter-particle interactions such as cross-linking between coatings.

### Substrate Material

In one or more embodiments of this invention the substrate and spacers can be made from, but not limited to, the following materials: polymer, metal, ceramic, semi-conductor, composite, carbohydrate, polysaccharide, polypeptide, protein, glycolipid, gel, or highly viscous glass. The materials can be molded, chemical vapor deposited, physical vapor deposited, ground, etched, solution precipitated, blown, vapor phase reacted, crushed, tumbled, polished, chemical mechanical planarized, electro discharge machined, pressed, stamped, lased, machined, poured, spun, pulled, pressed, welded, bonded, diffusion bonded, friction bonded, ultrasonically welded, ion beam welded, ion beam deposited, punched, pressure formed, gouged, cut, laser cut, abrasive blasted, freeze fractured, chemically foamed and cooled, cured, UV cured, photolithographied, 3D printed, stereolithographied, silk-screened, ink jet printed, fused, or made with any combination of the previously mentioned processes.

In one or more embodiments of this invention, the substrate and spacers are a polymer, and an example of the polymer is, but not limited to, silicone, polyethylene, polystyrene, polyuretane, polymethacrylate, polyester, and polycarbonate.

In one or more embodiments of this invention, the and spacers are a polymer film such as Mylar or woven or compressed polyester fabric.

### Immobilizing Agent

The immobilizing molecule (antibody or aptamer) can be adsorbed to the substrate or covalently bonded to the substrate. The large scale substrate is called the support substrate and the substrate can be of the same material of the support substrate or can be a different material as in the case of when the substrate is made of a material that coats the support substrate.

For adsorption, one option is to place the substrate in a liquid containing the aptamer or antibody for a long enough time so that they stick to the substrate. For proper sticking, the substrate needs to have a proper level of cleanliness.

For covalently bonding one option is to apply a metal, such as gold, to the support substrate and attach a thiol to both and in between the substrate and the aptamer or antibody. Another option is to attach a biotin to the aptamer or antibody and immobilize the biotin to the streptavidin that is on top of a gold surface.

Adsorbed Antibody onto Gold

Although from a bonding strength standpoint, covalent bonding of antibody to a substrate is stronger than relying upon adsorption of antibody onto a substrate, the inventor has discovered that for one or more embodiments of this invention, relying upon antibodies adsorbing onto gold can be robust enough for bacterial immobilization. This can simplify manufacturing process by avoiding the need to couple the antibodies covalently for making the immobilization particles. (Suo Z., et al. Efficient Immobilization and Patterning of Live Bacterial Cells. Langmuir 2008, 24, 4161-4167.)

Coating Substrate with Gold

For one or more embodiments of this invention, it is necessary to apply a layer of gold to the underlying substrate material. Prior to the application of the gold, the substrate may need to be cleaned appropriately, depending upon how clean the manufacturing step of the substrate was. If the underlying substrate is too dirty, the gold, or if necessary, preceding titanium layer, might not stick to the substrate and can fall off, thus reducing effective area of coverage for the antibodies. For one or more embodiments of this invention, a titanium or titanium/tungsten (20% titanium, 80% tungsten) layer of about 20 Angstroms is deposited prior to the application of gold. The titanium can be deposited with, but not limited to, a vacuum sputtering process, or other capable deposition process. For one or more embodiments of the invention, gold is deposited with a vacuum sputtering process. The gold should be deposited to approximately 100 Angstroms. If the gold is too thin, the gold layer might not form a continuous layer and will have islands of gold coverage which will provide less effective bonding area for the antibody. Although the gold can be made thicker than 100 Angstroms, but it is not necessary for one or more embodiments of this invention and will be less expensive to keep the layer to about 100 Angstroms.

For one or more embodiments of this invention, chromium is vacuum deposited (approx. 1 nm) followed by gold (approx. 39 nm)

### Maintaining Gold Cleanliness

Once the gold is deposited its surface should be kept clean enough not to interfere with the adsorption of antibody onto the gold. The gold surface can be kept clean with proper procedures to handle the substrate after deposition. This includes but not limited to High Efficiency Particulate Absorbing (HEPA) filtration of the air that comes into contact with the gold, transporting the gold coated substrates in a clean container, ensuring that workers that come into near contact with the gold coated substrates are wearing clean room gowns, facemasks, eye protection, and so on. If the gold surface becomes contaminated, it will require cleaning steps that are benign to the underlying substrate material.

### Cleaning Gold Surface

For one or more embodiments of this invention, the gold substrate is cleaned by wetting the gold with a boiling solution of H2O2 (35%), NH3 (25%) and Milli-Q water in a 1:1:5 ratio mixture for 10 minutes and rinsed thoroughly in Milli-Q water. (Schmid et al. Site-directed antibody immobilization on gold substrate for surface plasmon resonance sensors. Sensor and Actuators B: Chemical. Vol 113. Issue 1, 17 Jan. 2006, 297-303))

### Antibody Design—Immobilizing Microbes, Fungi, Phages, and Viruses

For one or more embodiments of this invention, the antibodies are optimized to immobilize the following, but not limited to, genus of bacteria for each of the mentioned diseases: Multiple Sclerosis—(Enterococcus, Streptococcus, Lactobacillus, Bacteroides, Escherichia, Clostridium, Serratia, Bifidobacterium, and Fusobacterium), ulcerative colitis—(Burkholderia, Mycobacterium, Bacillus, Clostridium, and Methylobacterium), Lupus—(Burkholderia, Mycobacterium, Pseudomonas, Methylobacterium, Vibrio, and Clostridium), Uveoretinitis—(Bacteriodes, Bacillus, Clostridium, Lactobacillus, Fusobacterium, Vibrio, Ruminococcus, Methylococcus), rheumatoid arthritis—gram positive bacteria. Removing these genus of bacteria are important as they can exacerbate symptoms.

For one or more embodiments of this invention, the antibodies are optimized to immobilize the following, but not limited to, genus of fungi: Saccharomyces and Candida.

For one or more embodiments of this invention, the antibodies are optimized to immobilize the following, but not limited to, virus: Influenza, Herpes, and Cytomegalovirus.

### Antibody Production

Standard techniques for producing monoclonal antibodies are used for target microorganisms. Microorganisms are selected from determining harmful microbes with methods that are outside the scope of this patent.

### Purification of Antibody

For one or more embodiments of this invention, the antibodies are purified by, but not limited to, the following methods: sodium sulphate precipitation (20% w/v) followed by Sephacryl S-200 HR gel filtration or protein A affinity chromatography (Van Erp, 1991, Van Erp et al., 1991 a).

### Acid Pretreatment of Antibody

For one or more embodiments of this invention, the inventor has discovered that pre-treating the antibodies with acid, or solution with a pH of approximately 1.0-3.0, can improve the binding capacity of the antibodies (van Erp, et al. Characterization of monoclonal antibodies physically adsorbed onto polystyrene latex particles. Journal of Immunological Methods. 152 (1992) 191-199.). For one or more embodiments of this invention the pretreatment can be, but not limited to, by mixing antibodies with 0.05M glycine/HCl buffer pH 2.0. After incubation of the antibody solution at 0-4 degrees Celsius for 1 hour, adjust the pH to 6.0-8.0 by the addition of 0.1 M NaOH.

### Cleaning Polymer

For one or more embodiments of this invention, the substrate is a polymer, such as but not limited to polystyrene, polyester, or nylon, and may require cleaning. If so the polymer surfaces can be cleaned with the following, but not limited to, cleaning solution. Make a phosphate buffer by mixing phosphate buffer powder (Wako Pure Chemical, Osaka, Japan) with ultra-pure water until a 1/15M solution and pH 7.4 is achieved. To clean the polymer surfaces, irrigate with the phosphate buffer solution and rinse with ultra-pure and/or deionized water. (Sato et al., Integration of an Immunosorbant Assay System Analysis of Secretory Human Immunoglobulin A on Polystyrene Beads in a Microchip. Anal Chem. 2000, 72,1144-1147.). Then dry polymer.

Adsorbed Antibody onto Polymer

Maximum adsorption of IgG antibody onto polymers have been observed to occur at pH 7 (Turkmen, et al. Phenylalanine Containing Hydrophobic Nanospheres for Antibody Purification. Biotechnol. Prog. 2008, 24, 1297-1303.)

For one or more embodiments of this invention that uses a polymer substrate, a method for adsorbing the antibodies, but not limited to, is described with following manner. Antibodies are added to a concentration of 0.001-10 mg/mL in 50 mmol/L carbonate buffer, pH 9.6 (coating buffer). Wet the polymer, by immersion, rinsing, or spraying, with the antibody mixture (described in this paragraph) and incubate for 8 hours at 4 degrees Celsius. Then rinse the polymer with deionized water to remove the antibody mixture. Dry polymer with nitrogen. (Qian, et al. Immobilization of Antibodies on Ultraflat Polystyrene Surfaces. Clinical Chemistry 46:9, 1456-1463. (2000).

For one or more embodiments of this invention, the polymer substrate can be, but not limited to polystyrene or polyester. (Boyd et al., Application of Antibody Adsorbed Polyester Cloth for Rapid Screening of Elution Conditions for Antigen Immunopurification. Immunological Investigations, Volume 25, Issue 5&6 September 1996, pages 447-453.)

### Covalent Bonding of Antibody

For one or more embodiments of this invention that uses gold as a substrate onto which the antibodies can be covalently bonded, a method for attaching antibodies, but not limited to, is described with the following manner. Wet the gold surface, by immersion, rinsing, or spraying, with a dilute (0.1-1.0 mM) ethanolic solution of dithiobis(succinimidyl undecanoate) (DSU) for 8-24 hours. Antibodies are added to a concentration of 0.5-2.0 mg/mL in 50 mM Delbucco's phosphate buffer (PBS) (Life Technologies) at pH 6.0. Wet the gold surface, by immersion, rinsing, or spraying, with the antibody mixture (described in this paragraph). Immerse in the antibody mixture for 30 minutes to 12 hours. 90 minutes is normally sufficient. (Mosher, et al. Microminiaturized Immunoassays Using Atomic Force Microscopy and Compositionally Patterned Antigen Arrays. Anal. Chem. 1998; 70, 1233-1241.)

For one or more embodiments of this invention, the inventor discovered that the following antibody attachment method covalently bond antibodies to the gold substrate. Wet gold substrate in 0.0002M dithiobissuccinimide propionate (DSP) prepared in dimethysulfoxide (DMSO) for 2 hours at room temperature. Then rinse with DMSO and then with phosphate buffered saline (PBS) at pH 7.4. (Schmid et al. Site-directed antibody immobilization on gold substrate for surface plasmon resonance sensors. Sensor and Actuators B: Chemical. Vol 113. Issue 1, 17 Jan. 2006, 297-303). Antibodies are added to a concentration of 0.5-2.0 mg/mL in 50 mM Delbucco's phosphate buffer (PBS) (Life Technologies) at pH 6.0. Wet the gold surface, by immersion, rinsing, or spraying, with the antibody mixture (described in this paragraph). Immerse in the antibody mixture for 30 minutes to 12 hours. 90 minutes is normally sufficient. (Mosher, et al. Microminiaturized Immunoassays Using Atomic Force Microscopy and Compositionally Patterned Antigen Arrays. Anal. Chem. 1998, 70, 1233-1241.)

For one or more embodiments of this invention, the inventor discovered that the following antibody covalent bonding method yields more antibodies that are properly oriented to immobilize the target microbe or proteins. Wet gold substrate in 0.0002M-0.0010M dithiobissuccinimide propionate (DSP) prepared in dimethysulfoxide (DMSO) for 2 hours at room temperature. Then rinse with DMSO and then with phosphate buffered saline (PBS) at pH 7.4. A Protein A layer is then covalently attached to the thiol linked gold substrate by soaking the gold substrate for 4-10 hours at 4 degrees Celsius in a Protein A solution (1 mg/ml) prepared in phosphate buffer. Then wet the gold substrate with ethanolamine hydrochloride (1 M), pH 8.6, for 1 hour to block the residual reacting sites. Then wash the substrate with distilled water and dry. (Schmid et al. Site-directed antibody immobilization on gold substrate for surface plasmon resonance sensors. Sensor and Actuators B: Chemical. Vol 113. Issue 1, 17 Jan. 2006, 297-303). Antibodies are added to a concentration of 0.5-2.0 mg/mL in 50 mM Delbucco's phosphate buffer (PBS) (Life Technologies) at pH 6.0. Wet the gold surface, by immersion, rinsing, or spraying, with the antibody mixture (described in this paragraph). Immerse in the antibody mixture for 30 minutes to 12 hours. 90 minutes is normally sufficient. (Mosher, et al. Microminiaturized Immunoassays Using Atomic Force Microscopy and Compositionally Patterned Antigen Arrays. Anal. Chem. 1998, 70, 1233-1241.)

### Covalent Bonding of Antibody to Polymer

One or more embodiments of the invention use antibodies that are covalently bonded to gold. The methods for accomplishing this are discussed in literature and are found in, but not limited to the following papers which are included by reference herein (Siiman et al. Covalently Bound Antibody on Polystyrene Latex Beads: Formation, Stability, and Use in Analysis of White Blood Cell Populations. Journal of Colloid and Interface Science 234, 44-58 (2001).).

Covalent Bonding Antibody with Thiol and Gold

One or more embodiments of the invention use antibodies that are covalently bonded to gold. The methods for accomplishing this are discussed in literature and are found in, but not limited to the following papers which are included by reference herein (Karyakin et al. Oriented Immobilization of Antibodies onto the Gold Surfaces via Their Native Thiol Groups. Anal. Chem., 2000, 72(16), pp 3805-3811.; Schmid et al. Site-directed antibody immobilization on gold substrate for surface plasmon resonance sensors. Sensor and Actuators B: Chemical. Vol 113. Issue 1, 17 Jan. 2006, 297-303)

Bonding Antibody with Biotin and Streptavidin deposited on Gold

### Antibody Repulsion Chemical

For one or more of the embodiments of this invention, octadecanethiol (ODT) can be used to prevent adhesion of antibodies.

### Aptamer Design

For one or more of the embodiments of this invention, the aptamers are of, but not limited to, the following kinds: DNA, RNA, and peptide. The SELEX, Complex Target SELEX, and Counter SELEX are processes used to identify the aptamers to use from a starting library of oligonucleotides and oligopeptides (approx 10̂5) that contain randomized regions. For one or more of the embodiments of this invention, it uses aptamer selection methods described literature, such as but not limited to SELEX, Complex Target SELEX, Counter SELEX, and methods contained in, but not limited to, the following parenthesized references (Tuerk, et al. Systematic evolution of ligands by exponential enrichment: RNA ligands to bacteriophage T4 DNA polymerase. Science. 3 Aug. 1990 249: 505-510, Shamah et al. Complex Target SELEX. Accounts of Chemical Research. 130-138 January 2008. Vol 41. No 1.; Homann et al. Combinatorial selection of high affinity RNA ligands to live African trypanosomes. Nucleic Acids Research, 1999, Vol 27, No. 9; Wang et al. In vitro selection of novel RNA ligands that bind human cytomegalovirus and block viral infection, RNA (2000) 6: 571-583; Keefe et al. SELEX with modified nucleotides. Current Opinion in Chemical Biology 2008. 12: 448-456; Hamula et al. Selection of Aptamers against Live Bacterial Cells. Anal. Chem. 2008, 80, 7812-7819; Hall et al. In Vitro Selection of RNA Aptamers to a Protein Target by Filter Immobilization. Current Protocols in Molecular Biology 24.3.1-24.3.27. October 2009) and those papers are incorporated by reference herein.

### Aptamer Design—Mimics

For one or more of the embodiments of this invention, the aptamers are optimized to immobilize certain autoimmune mimics. These mimics include, but are not limited to, tryptophan peptide from myelin basic protein (Westall, et al. 1971. Essential chemical requirements for induction of allergic encephalomyelitis. Nature 229: 22-24.), mid-region from myelin basic protein (Shapira, R, et al. 1971. Biological activity and synthesis of an encephalitogenic determinant. Science 172: 736-738.), hyperacute site from myelin basic protein (Westall, F. C, et al. 1977. Hyperacute autoimmune encephalomyelitis-unique determinant conferred by serine in a synthetic autoantigen. Nature 269: 425-427.), S-antigen 375-386 (Dua, H. S., et al. 1991. Structure-function studies of s-antigen: use of proteases to reveal a dominant uveitogenic site. Autoimmunity 10: 153-163.), acetylcholine receptor 129-145 (Yoshikawa, H., et al. 1997. A 17-mer self-peptide of acetylcholine receptor binds to B cell MHC class II, activates helper T cells, and stimulates autoantibody production and electrophysiologic signs of myasthenia gravis. J. Immunol. 159: 1570-1577.), acetylcholine receptor 67-75 (Bellone, M., et al. 1989. The main region of the nicotinic acetylcholine receptor. J. Immunol. 143: 3568-3579.), Sm b/B′ protein proline region (James, J. A., et al. 1999. Side-chain specificities and molecular modeling of peptide determinants for two anti-Sm B/B′ autoantibodies. J. Autoimmunity 12: 43-49.), and tropomyosin isomer V 4-10 (Vera, C., et al. 2000. Tropmodulin-binding site mapped to residues 7-14 at the N-terminal heptad repeats of tropomyosin isoform 5. Arch. Biochem. Biophys. 378: 16-24).

### Aptamer Design—Microbes

For one or more embodiments of this invention, the aptamers are optimized to immobilize the following, but not limited to, genus of bacteria for each of the mentioned diseases: Multiple Sclerosis—(Enterococcus, Streptococcus, Lactobacillus, Bacteroides, Escherichia, Clostridium, Serratia, Bifidobacterium, and Fusobacterium), ulcerative colitis—(Burkholderia, Mycobacterium, Bacillus, Clostridium, and Methylobacterium), Lupus—(Burkholderia, Mycobacterium, Pseudomonas, Methylobacterium, Vibrio, and Clostridium), Uveoretinitis—(Bacteriodes, Bacillus, Clostridium, Lactobacillus, Fusobacterium, Vibrio, Ruminococcus, Methylococcus), rheumatoid arthritis—gram positive bacteria. Removing these genus of bacteria are important as they can exacerbate symptoms.

For one or more embodiments of this invention, the aptamers are optimized to immobilize the following, but not limited to, genus of fungi: Saccharomyces and Candida.

For one or more embodiments of this invention, the aptamers are optimized to immobilize the following, but not limited to, virus: Influenza, Herpes, and Cytomegalovirus.

### Aptamer Design—Serpins

For one or more embodiments of this invention, the aptamers are optimized to immobilize serpins.

### Aptamer Selection Process—pH Effects

The gastrointestinal tract has a large range of pH from approximately 1 to 8 pH. For one or more of the embodiments of this invention, the inventor has discovered that when performing the aptamer selection process, SELEX, or its variants, the solution needs to be in the same range of pH that one would expect the binding to occur. For example, if the target microbe were to be found in the small intestine, it is important to select an aptamer to in a solution in an acidic environment of approximately 2-6 pH. For the large intestine, a pH of 7-8 should be the selection pH.

Aptamer Adsorbed onto Polymer

One or more embodiments of the invention use aptamers that are adsorbed onto polymer. The methods for accomplishing this—are discussed in literature and are found in, but not limited to the following paper which is included by reference herein (Balamurugan et al. Surface Immobilization Methods for aptamer diagnostic applications Analytical and Bioanalytical Chemistry vol 390 numb 4 February 2008).)

Aptamer Adsorbed onto Gold

One or more embodiments of the invention use aptamers that are adsorbed onto gold. The methods for accomplishing this, are discussed in literature and are found in, but not limited to the following paper which is included by reference herein (Wang et al. Aptamer biosensor for protein detection using gold nanoparticls Analytical Biochemistry vol 373 issue 2 February 2008).)

### Aptamer Covalently Bonded to Gold

One or more embodiments of the invention use aptamers that are covalently bonded to gold. The methods for accomplishing this, 5′ thiolated aptamer immobilized on gold, are discussed in literature and are found in, but not limited to the following paper which is included by reference herein (Savran et al. Micromechanical Detection of Proteins Using Aptamer-Based Receptor Molecules. Anal. Chem. 76, 3194-3198, (2004).)

One or more embodiments of the invention use aptamers that are covalently bonded to gold. The methods for accomplishing this, 5′ biotinylated aptamer immobilized on streptavidin fixed on gold surface with DSP, are discussed in literature and are found in, but not limited to the following paper which is included by reference herein (Liss et al., An Aptamer-Based Quartz Crystal Protein Biosensor. Anal. Chem., 74,4488-4495, (2002).)

[Aptamer Bonded to Streptavidin]

For one or more embodiments of this invention that uses gold as a substrate onto which the aptamers can be covalently bonded, a method for attaching aptamers, but not limited to, is described with the following manner.

Ideally the substrate material contains surfaces that will maintain the aptamer and/or antibodies some distance away from the body's surfaces so that the immune system will not react to the presence of the immobilized microorganisms or chemistries. So it will be essential to consider the maximum size of the immobilized organisms or chemicals to properly size the space between the immobilizing molecules and the minimum distance to the host's tissue surfaces, such as the mucosa of the gastrointestinal tract. One way of doing this is to have a concave or recessed surface. Another way is to have a porous surface. Another way is to have a filter like material with spacers at the outer surfaces to pull the filter-like material from the body's surfaces.

Immobilization Particle Design—Antibodies and/or Aptamers Adsorbed onto Porous Polymer Filter Paper as Substrate Support with Polymer Spacers
i. For one or more embodiments of this invention, the inventor has discovered that a woven, porous polymer, such as but not limited to polyester, polystyrene, nylon, fabric, paper, filter, or thin film (such as Mylar). Obtain a sheet of the polymer fabric, paper, filter, or thin film. Polyester films, such as Ultra-Polyester, are available in rolled films that are thicker than 1.5 microns. Limited by handling concerns during manufacturing, thinner is better because there is more surface to volume area available. For full production, the sheet of polymer can be on a roll and manufactured in a continuous process with the appropriate processing equipment.
ii. Clean polymer substrate according to the procedure described in the “Cleaning Polymer” section.
iii. adsorb immobilizing agent (aptamer or antibody) to the polymer as described in section “Adsorbed Antibody onto Polymer”
iv. make polymer spacers to prevent microbes or other immobilized body from communicating with the host immune system
   1. Inkjet and/or silkscreen tall (1 um-500 um), taller than the longest dimension of the intended immobilized body spaced 1 um-500 um apart, features or dots to both sides of fabric, paper, filter, or film substrate. The diameter of the features or dots should be approximately at a 1:1 ratio of diameter to their height to prevent breakage. The features, the spacers, can be made of the material mentioned earlier.
   2. Heat to form and deform, beyond the glass transition temperature, (with or without contact) polymer substrate while inactivating (denaturing, hiding, or destroying) any immobilization agent so that when the high spots touch the intestinal, or other bodily tissue, there is no immune response. The high spots need to be pushed in and out of the paper plane so that there are high spots on both side of the paper.
   3. Impact to form and deform with enough forces to inactivate any immobilization agent so that when the hot spots touch the intestinal, or other bodily tissue, there is no immune response. The high spots need to be pushed in and out of the paper plane so that there are high spots on both side of the paper.
v. Cut out pieces of the fabric, paper, filter, or film substrate, small enough that there is no chance of causing any digestive blockage if the immobilization particles are to be swallowed, 10 um-5000 um square or diameter. If the immobilization particle is be used as a dressing, then the fabric, paper, filter, or film substrate can be cut in larger pieces.
   1. For GI treatments, the cutting action should destroy any immobilizing agents at and slightly inbound from the perimeter of the cuts to prevent immobilizing bodies too close to the edge of the paper. The distance should be at least the largest dimension of the intended immobilized body. So if a bacteria is the target and it has a longest dimension of 5 microns, the immobilizing agents need to be destroyed up to around 5 microns from the edge.

Immobilization Particle Design—Antibodies and/or Aptamers Adsorbed to Concave Polymer Substrate Support
i. Obtain a silicon wafer (4″-12″ diameter), or other molding substrate if non-wafer manufacturing equipment and processes are used used.
ii. Spin on and bake photoresist onto the top, polished surface
iii. Prepare a photo mask with a grid pattern shown in FIG. XX
iv. Expose the photo resist coated wafer, or other molding substrate, in an appropriate stepper
v. Wet or dry etch to the proper depth of 0.01 um-500 um deep.
   1. For dry etching wafers, use an Applied Materials eMAX, Producer, or HART chamber. For other substrates, use the appropriate dry etch equipment.
   2. For wet etching, use standard semiconductor techniques for wet etching silicon, quartz, or other ceramic material of wafers or other molding substrates other than silicon are used
vi. Clean wafer or other molding substrate.
vii. Dry wafer or other molding substrate.
viii. Apply release chemical according to manufacturer's specification
ix. For one or more embodiments of this invention, the castable polymer can be, but not limited to, polyurethane. Pour on castable polymer.
x. Place sandwiching wafer onto the castable polymer and apply required pressure to ensure uniform thickness of the molded cavities
xi. Cure castable polymer according to manufacturer's specification
xii. Separate the silicon wafer from both sides of the cast polymer material
xiii. Clean cast polymer in appropriate solvents to remove release chemical. For example semiconductor grade acetone followed by semiconductor grade isopropyl alcohol
xiv. Apply a protective film, such as pressure sensitive adhesive kapton or PTFE film onto the flat, cavity-less side of the cast polymer material
xv. Apply adsorbed antibodies the surface of the cast polymer material that has cavities according to section “Adsorbed Antibody onto Polymer” (or aptamers) above.
xvi. Optional Step—place the cast polymer material, cavity-side down, onto an Applied Material Reflexion chemical mechanical planarization (CMP) pad of a CMP machine for 1-100 seconds. (platen speed 10-200 RPM, head rotation speed 10-200 RPM, head membrane pressure 0.2-20 PSI for each zone, 0.5-10 liter/min DI water with no slurry). This step removes any antibodies that are adsorbed outside of the cavity.
xvii. Remove the protective pressure sensitive adhesive film from the underside of the cast polymer material
xviii. Align the cast polymer material properly into a mounting support so that an excimer, infra-red, or near infra-red laser can cut each cavity into individual immobilization particles. Set the power density to 10-1,000,000 mW/cm̂2 and the linear feed rate from 0.1-1000 cm/sec.
xix. Laser cut each immobilization particle and separate.
   1. If necessary, for one or more embodiments of this invention, the immobilization particles can be cut by, but not limited to, sharp tool, electrical discharge, light, thermal, water saw, blasted abrasive materials, and other processes.

Immobilization Particle Design—Antibodies and/or aptamers Covalently (with Gold) Bonded onto Porous Polymer Filter Paper as Substrate Support with Polymer Spacers
i. For one or more embodiments of this invention, the inventor has discovered that a woven, porous polymer, such as but not limited to polyester, polystyrene, nylon, fabric, paper, filter, or thin film (such as Mylar). Obtain a sheet of the polymer fabric, paper, filter, or thin film. Polyester films, such as Ultra-Polyester, are available in rolled films as thin as 1.5 microns. Limited by handling concerns during manufacturing, thinner is better because there is more surface to volume area available. For full production, the sheet of polymer can be on a roll and manufactured in a continuous process with the appropriate processing equipment.
ii. Clean polymer substrate according to the procedure described in the “Cleaning Polymer” section.
iii. Sputter gold and other required metal films according to section “Coating substrate with Gold” onto the cavity side of the polyurethane material, 40-500 Angstroms thick. Use a standard sputtering tool, such as an Applied Materials PVD chamber with a gold and other metal targets. Use a standard sputtering process recipe. Turn the porous polymer filter paper sheet over to coat both sides with metal as needed.
iv. Apply covalently bonded antibodies to the gold surface deposited that was onto the polyurethane material that has cavities according to section “Covalent Bonding of Antibody” above.
v. make polymer spacers to prevent microbes or other immobilized body from communicating with the host immune system
   1. Inkjet tall, taller than the longest dimension of the intended immobilized body, dots to both sides of paper. The features, spacers, can be made of the same materials mentioned earlier.
   2. Silkscreen tall, taller than the longest dimension of the intended immobilized body, dots to both sides of paper.
   3. Heat to form and deform, beyond the glass transition temperature, (with or without contact) polymer substrate while inactivating (denaturing, hiding, or destroying) any immobilization agent so that when the high spots touch the intestinal, or other bodily tissue, there is no immune response. The high spots need to be pushed in and out of the paper plane so that there are high spots on both side of the paper.
   4. Impact to form and deform with enough forces to inactivate any immobilization agent so that when the hot spots touch the intestinal, or other bodily tissue, there is no immune response. The high spots need to be pushed in and out of the paper plane so that there are high spots on both side of the paper.
vi. Cut out pieces of the paper, small enough that there is no chance of causing any digestive blockage if the immobilization particles are to be swallowed. If to be used as a dressing, then can cut in larger pieces.
   1. For GI treatments, the cutting action should destroy any immobilizing agents at and slightly inbound from the perimeter of the cuts to prevent immobilizing bodies too close to the edge of the paper. The distance should be at least the largest dimension of the intended immobilized body. So if a bacteria is the target and it has a longest dimension of 5 microns, the immobilizing agents need to be destroyed up to around 5 microns from the edge.
   2. If necessary, for one or more embodiments of this invention, the immobilization particles can be cut by, but not limited to, sharp tool, electrical discharge, light, thermal, water saw, blasted abrasive materials, and other processes.

Immobilization Particle Design—Antibodies and/or Aptamers Covalently Bonded to Concave Polymer Substrate Support
i. Get a silicon wafer (4″-12″ diameter)
ii. Spin on and bake photoresist onto the top, polished surface
iii. Prepare a photo mask with a grid pattern shown in FIG. 6
iv. Expose the photo resist coated wafer in an appropriate stepper
v. Wet or dry etch to the proper depth of 0.01 um-500 um deep. The depth is based on an aspect ratio of depth:feature width of 0.001:1-5:1.
   1. For dry etching, use a standard silicon wafer etcher such as Applied Materials eMAX, Producer, or HART chamber with a standard dry etching process.
vi. Clean wafer
vii. Dry wafer
viii. Apply release chemical according to manufacturer's specification
ix. For one or more embodiments of this invention, the castable polymer can be, but not limited to, polyurethane. Pour on castable polymer.
x. Place sandwiching wafer onto the cast polymer and apply required pressure to ensure uniform thickness of the molded cavities
xi. Cure castable polymer according to manufacturer's specification
xii. Separate the silicon wafer from both sides of the cast polymer material
xiii. Clean cast polymer in appropriate solvents to remove release chemical. For example semiconductor grade acetone followed by semiconductor grade isopropyl alcohol
xiv. Sputter gold and other required metal films according to section “Coating substrate with Gold” onto the cavity side of the cast polymer material, 40-500 Angstroms thick. Use a standard sputtering tool, such as an Applied Materials Endura PVD chamber with a gold and other metal targets. Use a standard sputtering process recipe.
xv. Remove wafer from sputtering tool.
xvi. Apply a protective film, such as pressure sensitive adhesive kapton or PTFE film onto the flat, cavity-less side of the cast polymer material
xvii. Apply covalently bonded antibodies to the gold surface deposited that was onto the cast polymer material that has cavities according to section “Covalent Bonding of Antibody” above.
xviii. Optional Step—place the cast polymer material, cavity-side down, onto an Applied Material Reflexion chemical mechanical planarization (CMP) pad of a CMP machine for 1-100 seconds. (platen speed 10-200 RPM, head rotation speed 10-200 RPM, head membrane pressure 0.2-20 PSI for each zone, 0.5-10 liter/min DI water with no slurry). This step removes any antibodies that are adsorbed outside of the cavity.
xix. Remove the protective pressure sensitive adhesive film from the underside of the cast polymer material
xx. Align the cast polymer material properly into a mounting support so that an excimer, infra-red, or near infra-red laser can cut each cavity into individual immobilization particles. Set the power density to 10-1,000,000 mW/cm̂2 and the linear feed rate from 0.1-1000 cm/sec.
xxi. Laser cut each immobilization particle and separate.
   1. If necessary, for one or more embodiments of this invention, the immobilization particles can be cut by, but not limited to, sharp tool, electrical discharge, light, thermal, water saw, blasted abrasive materials, and other processes.

### Delivery Technology

Since one of the primary uses of one or more embodiments of this invention is for delivery of the immobilizing particles to the gastrointestinal tract, the immobilizing particles can be delivered orally or via the anus. For oral delivery a capsule, tablet, particle, or liquid form of delivery can work. Depending upon the location of the target microbes or chemicals that require immobilization, one or more embodiments of this invention can tailor the type of delivery method that is required.

### pH Delivery

For example, if the target region is the large intestine and one or more embodiments of this invention need to avoid deployment anywhere above the large intestine, encapsulating the immobilization particles inside a pH triggered capsule would be sufficient. One embodiment of such a capsule would be to place the immobilizing particles inside an open gel-cap and then close it with the opposing side of the gel-cap. Once closed, the gel-cap would get coated with a coating that dissolves when the pH of the environment reaches approximately 7. This is because the stomach's pH is approximately 1-2 (empty) and 3-4 (with food) due to the stomach acid and the pH gradually increases along the small intestine until it reaches approximately 7 at the entrance of the large intestine. Examples of a pH triggered coating are pharmaceutical grade shellac or Eudragit-S polymer. The standard way of coating the capsules is to place the capsules in a rotating coating pan where the coating is sprayed onto the capsules along with blowing hot dry air as they tumble inside the rotating coating pan. This tumbling and drying action is just like how clothing tumble inside the cylindrical rotating barrel of a clothes dryer.

### Coatings

If a treatment required a small intestinal delivery, one or more embodiments of this invention include a gel-cap that could survive the stomach acid and dissolve upon entry into the small intestine could be a satisfactory form of delivery. If a specific location in the small intestine was required, then a coating could be used and the coating's triggering pH can be tuned to open at the appropriate location in the small intestine. The coatings can be comprised of, but not limited to, a pH sensitive poly(meth)acrylate copolymer (Eudragit FS, Eudragit S(−100), Eudragit RL, Eudragit RS(−100), Eudragit L(−100)), ethylcellulose, shellac, deesterified pectin, polygalacturonic acid (PGA or its potassium and sodium salts), vinyl acetate resin, carboxylated polyvinyl acetates, polyvinyl/maleic anhydride copolymers, ethylene/maleic anhydride copolymers, methylacrylic acid/methyl methacrylate copolymers, waxes, and chitosan-calcium-alginate (Sriamornsak et. al. Composite Film-Coated Tablets Intended for Colon-Specific Delivery of 5-Aminosalicylic Acid: Using Deesterified Pectin. Pharm Dev and Tech. 2003, Vol. 8, No. 3, 311-318; Hua Z. et al. Technology to Obtain Sustained Release Characteristics of Drugs after Delivered to the Colon. Journal of Drug Targeting. Vol 6, Issue 6, July 1999, 439-448; Rudolph M. et al. A new 5-ASA multi-unit dosage form for the therapy of ulcerative colitis. European Journal of Pharmaceutics and Biopharmaceutics. Vol. 51, Issue 3, May 2001, 183-190; Gupta, et al. A novel pH- and time-based multi-unit potential colonic drug delivery system. I. Development. International Journal of Pharmaceutics. Vol. 213, Issues 1-2, 1 Feb. 2001, 83-91.; U.S. Pat. No. 5,401,512 Delayed release oral dosage forms for treatment of intestinal disorders).

Although many aptamers and antibodies can be stored in the dry condition on their substrate, some aptamers and antibodies may require an inert environment to protect from oxygen or other sources of harm. An inert purge gas such as nitrogen or argon can be used during the manufacture of the capsule. Also, a dry mixture of minerals and/or vitamins can be added to the immobilization particles.

### Mineral Supplement

Examples of such minerals are but not limited to magnesium, selenium, manganese, iron, chromium, calcium, iodine, chloride, sodium, potassium, boron, bromide, silicon, phosphorus, titanium, rubidium, cobalt, copper, antimony, molybdenum, strontium, zinc, nickel, tungsten, scandium, vanadium, tellurium, tin, lanthanum, yttrium, silver, gallium, bismuth, zirconium, cerium, cesium, gold, beryllium, hafnium, samarium, terbium, europium, gadolinium, dysprosium, thorium, holmium, lutetium, erbium, ytterbium, neodymium, praseodymium, niobium, tantalum, thallium, rhenium, indium, etc.. These elements can be added by using a trace mineral powder complex manufactured by Trace Minerals Research of Ogden, Utah. The minerals can also be added individually or in mixtures by powders supplied by many nutritional supplement ingredient companies.

### Packaging

For one or more embodiments of this invention the immobilization particles can be packaged into a sachet for easy and convenient storage and use and later eaten alone or with food. For one or more embodiments of this invention the immobilization particles can be delivered in a cap of a drink bottle where the patient breaks a seal and mixes the particles into the drink prior to consumption. For one or more embodiments of this invention the immobilization particles can be premixed in a liquid drink or foodstuff.

### Coated Immobilization Particles

For one or more embodiments of this invention the immobilization particles can have enteric coatings individually or for clusters of immobilization particles. This way the immobilization particles can be ingested in a non-capsule or non-tablet form and be delivered at a specific location in the small intestine or large intestine. For one or more embodiments of this invention the enteric coated immobilization particles can be packaged into a sachet for easy and convenient storage and use and later eaten alone or with food. For one or more embodiments of this invention the enteric coated immobilization particles can be delivered in a cap of a drink bottle where the patient breaks a seal and mixes the particles into the drink prior to consumption. For one or more embodiments of this invention the enteric coated immobilization particles can be premixed in a liquid drink or foodstuff. The coatings can be comprised of, but not limited to, a pH sensitive poly(meth)acrylate copolymer (Eudragit FS, Eudragit S(−100), Eudragit RL, Eudragit RS(−100), Eudragit L(−100)), ethylcellulose, shellac, deesterified pectin, polygalacturonic acid (PGA or its potassium and sodium salts), vinyl acetate resin, carboxylated polyvinyl acetates, polyvinyl/maleic anhydride copolymers, ethylene/maleic anhydride copolymers, methylacrylic acid/methyl methacrylate copolymers, waxes, and chitosan-calcium-alginate (Sriamornsak et. al. Composite Film-Coated Tablets Intended for Colon-Specific Delivery of 5-Aminosalicylic Acid: Using Deesterified Pectin. Pharm Dev and Tech. 2003, Vol. 8, No. 3, 311-318; Hua Z. et al. Technology to Obtain Sustained Release Characteristics of Drugs after Delivered to the Colon. Journal of Drug Targeting. Vol 6, Issue 6, July 1999, 439-448; Rudolph M. et al. A new 5-ASA multi-unit dosage form for the therapy of ulcerative colitis. European Journal of Pharmaceutics and Biopharmaceutics. Vol. 51, Issue 3, May 2001, 183-190; Gupta, et al. A novel pH- and time-based multi-unit potential colonic drug delivery system. I. Development. International Journal of Pharmaceutics. Vol. 213, Issues 1-2, 1 Feb. 2001, 83-91.; U.S. Pat. No. 5,401,512 Delayed release oral dosage forms for treatment of intestinal disorders). Since the patient can eat one or more embodiments of the invention in this form of delivery, it helps the patient feel like the medication is a food product rather than pill form of medicine. This has a psychological benefit which can improve patient compliance. In one or more embodiments of this invention there can be a flavored coating over the enteric coating to further disguise the medication as a food product to further encourage compliance.

### Liquid Delivery

For one or more embodiments of this invention the immobilization particles can be delivered in a mixture with a non-toxic liquid such as a food based oil or water based saline solution inside a sealed capsule. Along with the liquid the appropriate preservative chemicals such as antioxidants can be added. Examples of anti-oxidants that can be added, but not limited to, are vitamin C, vitamin E, alpha-lipoic acid, uric acid, selenium, carotenoids, super oxide dismutase, resveratrol, and pycnogenol. Medicinal herbs can also be added to the liquid. Examples of medicinal herbs that can be added, but not limited to, aloe vera, Cat's Claw, Echinacea, and Golden Seal.

### Anal Delivery

For one or more embodiments of the invention the immobilization particles can be delivered via the anus in a suppository or enema form. The suppository can be a capsule containing immobilization particles. For an enema, the immobilization particles would be carried in a liquid. The immobilization particles and liquid can be stored in a plastic bottle until the enema would be ready for use. A nozzle on the plastic bottle would permit comfortable and safe delivery of the liquid and immobilization particles into the lower large intestine.

For one or more embodiments of the enema, various chemicals can be added to help treat an illness. Examples of such chemicals that can be added, but not limited to, are butyric acid, bismuth-containing compounds, alpha-lipoic acid, super oxide dismutase, Vitamin E, Vitamin C, Cat's Claw, and aloe vera.

### Topical Delivery

For one or more embodiments of this invention the immobilization particles can be applied topically to control infection or the microbiome on the skin, nasal and sinus cavity, urogenital, ear, and or vaginal tract. For one or more embodiments of this invention the immobilization particles can be mixed into a topical cream or gel. For one or more embodiments of this invention the immobilization particles can be mixed into an irrigation liquid or gas.

For one or more embodiments of this invention the immobilization particles can be embedded into a bandage to cover a wound or to locally control the microbiome.

If an oil is used as a liquid, probiotics, such as but not limited to Lactobacillus

1. A pharmaceutical compound where a binding molecule is attached to a substrate that can be consumed.
